(19) United States
(12) Patent Application Publication
(10) Pub. No.: US 2005/0136026 A1
(43) Pub. Date: Jun. 23, 2005

(54) Ordered liquid crystalline cleansing composition with C16-24 normal monoalkylsulfosuccinates and C16-24 normal alkyl carboxylic acids

(76) Inventors: Qiang Qiu, Harrison, NJ (US); Frank Meyer, Fort Lee, NJ (US); Rajendra M. Dave, Newark, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/744,749

(22) Filed: Dec. 23, 2003

(51) Int. Cl. A61K 7/075; A61K 7/08; A61K 9/00

(52) U.S. Cl. 424/70.24

(57) ABSTRACT

An ordered liquid crystalline phase cleansing composition is disclosed that contains an anionic surfactant and a structuring base containing a C16 to C24 normal monoalkylsulfosuccinate and a C16 to C24 normal alkyl fatty acid with an alkyl carbon number that differs by 4 carbons or less compared to the monoalkylsulfosuccinate and that is in a specified mole ratio range.

### BACKGROUND OF THE INVENTION

1. Field of the Invention

The present invention relates to detergent compositions suitable for topical application for cleansing the human body, such as the skin and hair. In particular, it relates to ordered liquid crystalline phase compositions containing long chain normal monoalkylsulfosuccinates.

2. Background of the Art

In order to be acceptable to consumers, a liquid personal cleansing product must exhibit good cleaning properties, must exhibit good lathering characteristics, must be mild to the skin (not cause drying or irritation) and preferably have user desired product viscosity while maintaining the rinse/after-feel characteristics of the active components.

The majority of prior art liquid cleansing products contain polymers, long-chain unneutralized fatty acids (e.g. C16-C18) or oils as thickeners to increase product viscosity. Although these materials might produce the desired viscosity, they structure the product such that its kinetics of dispersion is slow where the lather is slow to build or slow to rinse off. As a consequence, such products suffer from poor lather or slimy rinse or lack of clean/fresh feel (i.e. a lack of a squeaky clean feel) after towel-drying. In extreme cases where high amounts of structurants are desired, the product usually becomes very difficult to disperse in water and very slow to rinse off. Surprisingly it has been found that the use of an ordered liquid crystalline phase cleansing composition preferably containing swollen lamellar gels formed by C16-24 normal monoalkylsulfosuccinates in combination with normal alkyl fatty acids having an alkyl carbon numberin the same range, and advantageously within 4 carbons, has the advantage of fast dispersion kinetics (with the same product viscosity), while ensuring high product viscosity. Such systems are unexpectedly found to be fast in releasing active ingredients (i.e. lather-forming surfactants) and improving the rinse and clean/fresh feeling accompanying product use.

Combinations of sulfosuccinates and fatty acids having various alkyl chain lengths have been disclosed in liquid cleansing compositions. For example, PCT/WO publication WO 98/04233 published on Feb. 5. 1998 discloses conditioning shampoos that may contain a wide range of mono and dialkyl sulfosuccinates in combination with C16-22 alkyl fatty acids. Similarly U.S. Pat. No. 6,001,787 issued to Pratley on Dec. 14, 1999 discloses an aqueous cleansing composition that may include C13-16 alkyl sulfosuccinates and C6-11 fatty acids. U.S. Pat. No. 4,435,300 issued to Guth et al. On Mar. 6, 1984 discloses cleansing compositions that may contain C₈₋₁₇ alkyl sulfosuccinates and C5-17 fatty acids. U.S. Pat. No. 5,424,010 issued to Duliba et al. On Jun. 13, 1995 discloses a dish washing composition that contains C4-22 dialkyl sulfosuccinates. However there is no disclosure or suggestion in any of these patents and patent applications of an ordered liquid crystal cleansing composition having a specific mole ratio of a C16 to 24 normal monoalkylsulfosuccinate to a normal alkyl fatty acid within the same alkyl carbon number range and advantageously within 4 carbons.

### BRIEF DESCRIPTION OF THE INVENTION

In one aspect of the present invention is an ordered liquid crystalline cleansing composition including but not limited to:
a. at least 3% by wt. of an anionic surfactant other than a C16 to C24 normal monoalkylsulfosuccinate;
b. a structuring base including at least one compound selected from C16 to C24 normal monoalkylsulfosuccinates and at least one compound selected from C16 to C24 normal alkyl fatty acids;
c. wherein the at least one monoalkylsulfocuccinate has an alkyl group with a carbon number n and the at least one normal fatty acid has an alkyl group with a carbon number m and the absolute value of n−m is 4 or less; and
d. wherein the mole ratio of said at least one monoalkylsulfosuccinate to said at least one fatty acid is in the range of about 3 to 0.2.

### BRIEF DESCRIPTION OF THE FIGURES

FIG. 1 illustrates the comparison of the XRD spectrograms of samples C to G of sodium mono-stearoyl sulfosuccinate at different mixing ratios with stearic acid as described Example 1.

### DETAILED DESCRIPTION OF THE INVENTION

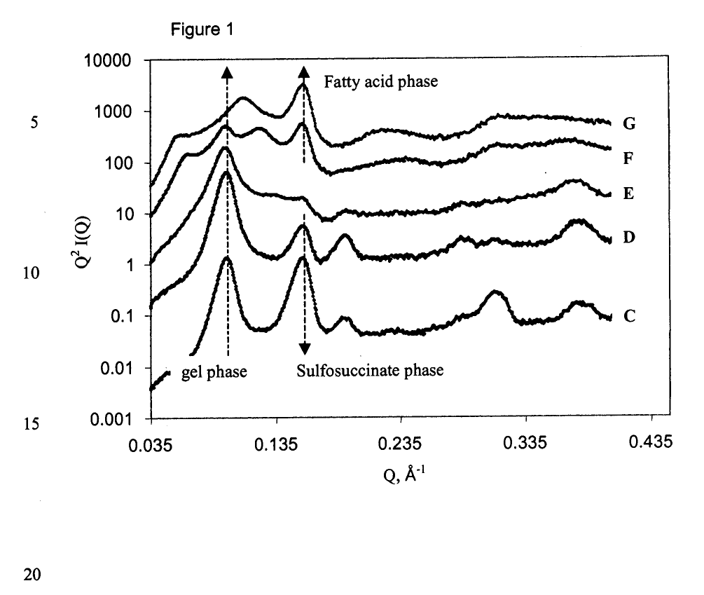

FIG. 1 shows that as the mole ratio of sulfosuccinate to stearic acid changes from 2:1 to 1:2, the formation of a sulfosuccinate/stearic acid gel phase is enhanced while the sulfosuccinate-only phase gradually disappears. From mole ratio 1:2 to 1:5, there is the formation of stearic acid-only phase.

In one aspect of the invention is an ordered liquid crystalline phase cleansing composition including but not limited to:
a. at least 3% (preferably 4, 5, 7, 10, 11, 15, 20, 21, 25, or 30%) by wt. of an anionic surfactant other than a C16 to C24 normal monoalkylsulfosuccinate;
b. a structuring base including at least one compound selected from C16 to C24 normal monoalkylsulfosuccinates (preferably C16 to C18) and at least one compound selected from C16 to C24 normal alkyl fatty acids (preferably C16 to C18);
c. wherein the at least one monoalkylsulfocuccinate has an alkyl group with a carbon number n and the at least one normal fatty acid has an alkyl group with a carbon number m and the absolute value of n−m is 4 or less (preferably 2 or less, most preferably 0); and
d. wherein the mole ratio of said at least one monoalkylsulfosuccinate to said at least one fatty acid is in the range of about 3 to 0.2 (preferably about 2.5 to 0.3, more preferably about 2 to 0.4, most preferably about 1.5 to 0.5, and advantageously about 1.2 to 0.5).

Advantageously, the at least one normal monoalkylsulfosuccinate is present in a concentration of at least about 1% (preferably at least 2 or 3%) by wt.). Preferably the inventive cleansing composition has a specific dispersion rate of at least about 40 mls when the viscosity of the composition is greater than about 100 Pa S at 25 C measured at a shear rate of 0.1 sec⁻¹ according to the Standard viscosity method (provided below). Advantageously the pH of the composition is less than 8.0, preferably between 4.5 and 7.5, even more preferably between 5.5 and 7.0. The solubilizing cation of the at least one monoalkylsulfosuccinate is advantageously selected from sodium, potassium, ammonium, alkanol ammonium or a blend thereof. The inventive composition preferably has a specific water loss of less than about 10% by wt., more preferably less than about 5% by wt.

C16-C24 Monoalkylsulfosuccinates:

Of the inventive C16-C24 Monoalkylsulfosuccinates which are synthesized by art recognized techniques, the C12-18 normal alkyl sulfosuccinates that were used in the examples were characterized as follows:

C 12/14 have the following alkyl chain distribution—C10:<2%; C12: 67-75%; C14: 20-30%; C 16:<2%.

C 16 has the following alkyl chain distribution—C14:<2.5%; C16:>95%; C18:<5%.

C 18 has the following alkyl chain distribution—C16:<3%; C18:>97%; C20:<3%.

Surfactants:

Surfactants are an essential component of the inventive cleansing composition. They are compounds that have hydrophobic and hydrophilic portions that act to reduce the surface tension of the aqueous solutions they are dissolved in. Useful surfactants include anionic, surfactants and optionally nonionic, amphoteric, and cationic surfactants, and blends thereof.

Anionic Surfactants:

The cleansing composition of the present invention contains one or more anionic detergents other than C16-24 normal monoalkylsulfusuccinates. The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably greater than 3; and M is a
solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₁₄ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:
R⁴O₂CCH₂CH(SO₃M)CO₂M; and
amide-MEA sulfosuccinates of the formula;
   R⁴CONHCH₂CH₂O₂CCH₂CH(SO₃M)CO₂M
wherein R⁴ ranges from C₈-C₂₂ alkylexcept for monoalkyl sulfosuccinates where R⁴ is C₈₋₁₄ and M is a solubilizing cation.

Sarcosinates are generally indicated by the formula:
R¹CON(CH₃)CH₂CO₂M,
wherein R¹ ranges from C₈-C₂₀ alkyl and M is a solubilizing cation.

Taurates are generally identified by formula:
R²CONR³CH₂CH₂SO₃M
wherein R² ranges from C₈-C₂₀ alkyl, R³ ranges from C₁-C₄ alkyl and M is a solubilizing cation.

The inventive cleansing composition contains anionic surfactants, preferably C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

The acyl isethionate may be an alkoxylated isethionate such as is described in Ilardi et al., U.S. Pat. No. 5,393,466, titled “Fatty Acid Esters of Polyalkoxylated isethonic acid; issued Feb. 28, 1995; hereby incorporated by reference. This compound has the general formula: 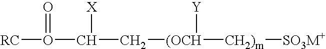
wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Amphoteric Surfactants

One or more amphoteric surfactants may be used in this invention. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula: 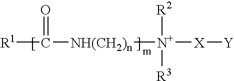
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula: 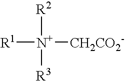
and amido betaines of formula: 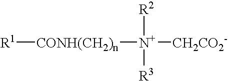
where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula: 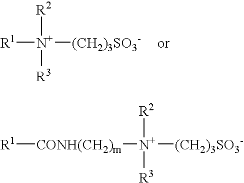
where m is 2 or 3, or variants of these in which —(CH₂)₃ SO₃— is replaced by

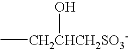

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like. Amphoteric surfactants are preferably used at a minimum level of about 1% by wt. and preferably at a maximum level of about 10% by wt.

Nonionic Surfactants

One or more nonionic surfactants may also be used in the cleansing composition of the present invention.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in Patent No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference. Nonionic surfactants are preferably used at a minimum level of about 0.5% by wt. and preferably at a maximum level of about 20% by wt.

Cationic Skin Conditioning Agents

An optional component in compositions according to the invention is a cationic skin feel agent or polymer, such as for example cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation. Cationic polymers are preferably used at a minimum level of abou 0.05% by wt. and at a maximum level of about 0.5% by wt.

Cationic Surfactants

One or more cationic surfactants may also be used in the cleansing composition. Cationic surfactants are preferably used at a minimum level of about 0.1% by wt. and at a maximum level of about 5% by wt.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued Mar., 27, 1973; and “Surface Active Agents and Detergents” (Vol. I & II) by Schwartz, Perry & Berch, both of which are also incorporated into the subject application by reference.

In addition, the inventive cleansing composition of the invention may include 0 to 15% by wt. optional ingredients as follows: perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise antimicrobials such as 2-hydroxy-4,2′, 4′ trichlorodiphenylether (DP300); preservatives such as dimethyloldimethylhydantoin (Glydant XL1000), parabens, sorbic acid etc., and the like.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

Emollients:

Hydrophillic emollients or humectants such as polyhydric alcohols, e.g. glycerine and propylene glycol, and the like; and polyols such as the polyethylene glycols listed below and the like may be used.

| | |
|---|---|
| Polyox WSR-205 | PEG 14M, |
| Polyox WSR-N-60K | PEG 45M, or |
| Polyox WSR-N-750 | PEG 7M. |

A blend of a hydrophobic and hydrophilic emollients may be used. Preferably, hydrophobic emollients are used in excess of hydrophilic emollients in the inventive cleansing composition. Most preferably one or more hydrophobic emollients are used alone. Hydrophobic emollients are preferably present in a concentration greater than about 10% by weight, more preferably about 12% by weight. The term “emollient” is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

Useful emollients include the following:
(a) silicone oils and modifications thereof such as linear and cyclic polydimethylsiloxanes; amino, alkyl, alkylaryl, and aryl silicone oils;
(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;
(c) waxes such as carnauba, spermaceti, beeswax, lanolin, and derivatives thereof;
(d) hydrophobic and hydrophillic plant extracts;
(e) hydrocarbons such as liquid paraffins, vaseline, microcrystalline wax, ceresin, squalene, pristan and mineral oil;
(f) higher fatty acids such as lauric, myristic, palmitic, stearic, behenic, oleic, linoleic, linolenic, lanolic, isostearic, arachidonic and poly unsaturated fatty acids (PUFA);
(g) higher alcohols such as lauryl, cetyl, stearyl, oleyl, behenyl, cholesterol and 2-hexydecanol alcohol;
(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;
(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils;
(j) lipids such as cholesterol, ceramides, sucrose esters and pseudo-ceramides as described in European Patent Specification No. 556,957;
(k) vitamins, minerals, and skin nutrients such as milk, vitamins A, E, and K; vitamin alkyl esters, including vitamin C alkyl esters; magnesium, calcium, copper, zinc and other metallic components;
(l) sunscreens such as octyl methoxyl cinnamate (Parsol MCX) and butyl methoxy benzoylmethane (Parsol 1789);
(m) phospholipids;
(n) antiaging compounds such as alpha hydroxy acids, beta hydroxy acids; and
(o) mixtures of any of the foregoing components, and the like.

Preferred emollients are selected from triglyceride oils, mineral oils, petrolatum, and mixtures thereof. Further preferred emollients are triglycerides such as sunflower seed oil.

Ordered Liquid Crystalline Compositions:

The inventive cleansing composition possesses ordered liquid crystalline microstructure, preferably lamellar microstructure. The rheological behavior of all surfactant solutions, including liquid cleansing solutions, is strongly dependent on the microstructure, i.e., the shape and concentration of micelles or other self-assembled structures in solution.

When there is sufficient surfactant to form micelles (concentrations above the critical micelle concentration or CMC), for example, spherical, cylindrical (rod-like or discoidal), spherocylindrical or ellipsoidal micelles may form. As surfactant concentration increases, ordered liquid crystalline phases such as lamellar phase, hexagonal phase, cubic phase or L3 sponge phase may form. The lamellar phase, for example, consists of alternating surfactant bilayers and water layers. These layers are not generally flat but fold to form submicron spherical onion like structures called vesicles or liposomes. The hexagonal phase, on the other hand, consists of long cylindrical micelles arranged in a hexagonal lattice. In general, the microstructure of most personal care products consist of either spherical micelles; rod micelles; or a lamellar dispersion.

As noted above, micelles may be spherical or rod-like. Formulations having spherical micelles tend to have a low viscosity and exhibit Newtonian shear behavior (i.e., viscosity stays constant as a function of shear rate; thus, if easy pouring of product is desired, the solution is less viscous and, as a consequence, it doesn't suspend as well). In these systems, the viscosity increases linearly with surfactant concentration.

Rod micellar solutions are more viscous because movement of the longer micelles is restricted. At a critical shear rate, the micelles align and the solution becomes shear thinning. Addition of salts increases the size of the rod micelles thereof increasing zero shear viscosity (i.e., viscosity when sitting in bottle) which helps suspend particles but also increases critical shear rate (point at which product becomes shear thinning; higher critical shear rates means product is more difficult to pour).

Lamellar dispersions differ from both spherical and rod-like micelles because they can have high zero shear viscosity (because of the close packed arrangement of constituent lamellar droplets), yet these solutions are very shear thinning (readily dispense on pouring). That is, the solutions can become thinner than rod micellar solutions at moderate shear rates.

In formulating liquid cleansing compositions, therefore, there is the choice of using rod-micellar solutions (whose zero shear viscosity, e.g., suspending ability, is not very good and/or are not very shear thinning); or lamellar dispersions (with higher zero shear viscosity, e.g. better suspending, and yet are very shear thinning). Such lamellar compositions are characterized by high zero shear viscosity (good for suspending and/or structuring) while simultaneously being very shear thinning such that they readily dispense in pouring. Such compositions possess a “heaping”, lotion-like appearance which convey signals of enhanced moisturization.

When rod-micellar solutions are used, they also often require the use of external structurants to enhance viscosity and to suspend particles (again, because they have lower zero shear viscosity than lamellar phase solutions). For this, carbomers and clays are often used. At higher shear rates (as in product dispensing, application of product to body, or rubbing with hands), since the rod-micellar solutions are less shear thinning, the viscosity of the solution stays high and the product can be stringy and thick. Lamellar dispersion based products, having higher zero shear viscosity, can more readily suspend emollients and are typically more creamy. In general, lamellar phase compositions are easy to identify by their characteristic focal conic shape and oily streak texture while hexagonal phase exhibits angular fan-like texture. In contrast, micellar phases are optically isotropic.

It should be understood that lamellar phases may be formed in a wide variety of surfactant systems using a wide variety of lamellar phase “inducers” as described, for example, in U.S. Pat. No. 5,952,286 issued to Puvvada, et al., on Sep. 14, 1999. Generally, the transition from micelle to lamellar phase are functions of effective average area of headgroup of the surfactant, the length of the extended tail, and the volume of tail. Using branched surfactants or surfactants with smaller headgroups or bulky tails are also effective ways of inducing transitions from rod micellar to lamellar.

One way of characterizing ordered liquid crystalline dispersions include measuring viscosity at low shear rate (using for example a Stress Rheometer) when additional inducer (e.g., oleic acid or isostearic acid) is used. At higher amounts of inducer, the low shear viscosity will significantly increase.

Another way of measuring ordered liquid crystalline dispersions is using freeze fracture electron microscopy. Micrographs generally will show ordered liquid crystalline microstructure and close packed organization of the lamellar droplets (generally in size range of about 2 microns).

The inventive ordered liquid crystalline-isotropic composition preferably has a low shear viscosity in the range of about 10 to 1000 Pa S measured at 0.1 S⁻¹ using SR-5 stress controlled rheometer using the procedure described below. More preferably the viscosity range is about 50 to 500 Pa S.

The invention will now be described in greater detail by way of the following non-limiting examples. The examples are for illustrative purposes only and not intended to limit the invention in any way.

Except in the examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

All percentages in the specification and examples are intended to be by weight unless stated otherwise.

### EXAMPLE 1

X-ray diffraction studies were undertaken according to the method described below of samples A-G, whose compositions are given in Table 1. Representative X-ray spectrums are given in FIG. 1 for samples C-G (A & B have features like C-D, and are omitted for figure clarity).

The XRD results indicate the formation of a gel phase when C18 sulfosuccinate is mixed with C18 fatty acid. Preferably a critical ratio of about 1:1.2, in terms of weight, or 1:2 mole ratio, of sulfosuccinate to fatty acid is required for the formation of gel phase to reach maximum as illustrated in FIG. 1 (sample E). Below this ratio, there is coexistence of sulfosuccinate crystals and sulfosuccinate/fatty acid gels as illustrated in FIG. 1 (samples C & D), and above this ratio, coexistence of free fatty acids and sulfosuccinate/fatty acid gel phase as illustrated in FIG. 1 (samples F & G). Since this ratio is close to the known acid/soap stoichiometry (e.g. see J. Phys. Chem., V.100, 357-361, 1996) of long chain fatty acids, it is deduced that the sulfosuccinate material used in this study is relatively pure, i.e. there is no significant presence of fatty alcohols as impurities.

### EXAMPLE 2

In accordance with the XRD results, observation revealed that at a given overall concentration (1.33 moles/L) different mixing ratios of sulfosuccinate and fatty acid affect differently their structuring capability of the aqueous solution. In Table 1 is shown the water-bonding capacity of the sulfosuccinate/fatty acid combinations A to G, that are the same set of samples used in XRD study. At the studied concentrations, free water phase was seen in the sulfosuccinate-rich solutions (A-D), i.e. before reaching the critical mole ratio 1:2 where formation of gel phase reaches a maximum (E). This free water has a higher activity than water bound to the gel or crystal phase, thus accounting for the faster weight loss of the corresponding solutions. Although no free water phase was observed in the fatty acid-rich phase (F-G), it is clear that water loss is also significant at the higher phase ratio of fatty acid to gel (F). The ones exhibiting minimum water loss are those with a mole ratio of approx. 1:2.

TABLE 1

| | | | | | % water | % water loss | % water loss |
|---|---|---|---|---|---|---|---|
| Sample | C18 SS* | C18 FA* | Mole ratio: | Appearance | loss at 75 C. | at 75 C. | at 75 C. |
| Code | Wt. % | Wt. % | SS:FA | at 22 C. | 10 mins | 30 mins | 60 mins |
| A | 34 | 4 | 5:1 | Free water on top | 2.0 | 5.7 | 10.1 |
| B | 30.5 | 6 | 3:1 | Free water on top | 1.6 | 3.7 | 5.4 |
| C | 28 | 8.2 | 2:1 | Free water on top | 1.0 | 4.2 | 7.5 |
| D | 21 | 11.8 | 1:1 | Free water on top | 0.5 | 2.3 | 4.7 |
| E | 13.8 | 16 | 1:2 | Homogneneous | 0.6 | 1.8 | 2.8 |
| F | 10.2 | 18 | 1:3 | Homogeneous | 0.5 | 1.7 | 2.8 |
| G | 7 | 20 | 1:5 | Homogeneous | 0.7 | 2.4 | 3.8 |

*Note: the overall weight is taken such that the overall number of moles of sulfosuccinate and fatty acid remains constant to facilitate comparison of molecular structuring capacity.

### EXAMPLE 3

A study of viscosity as a function of mole ratio in C12 surfactant solution samples H to L was also performed as illlustrated in table 2. A dilute surfactant solution—10% wt. Sodium Cocoyl Isethionate (SCI) was mixed with different combinations of normal monoalkylsulfosuccinate (SS) and fatty acids—12% wt. in total.

The results are summarised in table 2. The results show that, within the range studied, the solution is best structured between the ratio 2.3:1 and 1:1, in terms of weight ratio, or between 1.4:1 and 1:1.7, in terms of mole ratio.

TABLE 2

| | | | | | | | | Viscosity at 0.1 S⁻¹ | |
|---|---|---|---|---|---|---|---|---|---|
| | | | | | | | Mole | and 25 C. Pa S | |
| Sample | SCI | C16 SS | C18 SS | C16FA | C18FA | Weight Ratio: | Ratio: | (appearance) | |
| Code | % wt. | % wt. | % wt. | % wt. | % wt. | SS:FA | SS:FA | 4 days | 42 days |
| H | 10 | 4.8 | 4.8 | 1.2 | 1.2 | 4:1 | 2.4:1 | (free flow | 132 |
| | | | | | | | | liquid) | |
| I | 10 | 4.2 | 4.2 | 1.8 | 1.8 | 2.3:1 | 1.4:1 | 38 | 339 |
| | | | | | | | | (paste) | |
| J | 10 | 3.8 | 3.8 | 2.2 | 2.2 | 1.7:1 | 1:1 | 70 | 878 |
| | | | | | | | | (paste) | |
| K | 10 | 3.0 | 3.0 | 3.0 | 3.0 | 1:1 | 1:1.7 | 18 | 143 |
| | | | | | | | | (paste) | |
| L | 10 | 2.0 | 2.0 | 4.0 | 4.0 | 1:2 | 1:3.4 | (free flow | 26 |
| | | | | | | | | liquid) | |

### EXAMPLE 4

Dispersion kinetics or lathering rate of the formulations described in Table 3 were studied using a rotating cylinder test (see method below). In this test, 5.5 g formulation amounts were prepared according to the procedure below and transferred into the bottom of the cylinder, then 30 g distilled water was carefully added into the cylinder. Caution was taken to avoid any pre-dispersion of the product, nor any foaming prior to cylinder rotation. Thus, the speed of lather, i.e. lather volume at different number of rotations, is primarily determined by the availability of surfactants, SCI in this case, in the water phase as it is gradually released from the formulation. The results indicate the high lather rate (i.e. high dispersion kinetics) found for the inventive sulfosuccinates/fatty acids (J) combination compared to the comparative cases (M and N).

TABLE 3

| | | Foam Volume at different number | | | | |
|---|---|---|---|---|---|---|
| | | of rotations | | | | |
| | Formulation in | Data normalized in ml/g_product | | | | |
| Sample Code | wt. % | 5 | 20 | 40 | 60 | 90 |
| J | Inventive: | 5 | 28 | 44.4 | 51 | 60.9 |
| | 10% SCI, | | | | | |
| | 3.8% C16SS, | | | | | |
| | 3.8% C18SS, | | | | | |
| | 2.2% C16FA, | | | | | |
| | 2.2% C18FA | | | | | |
| M | Comparative A: | 3.3 | 9.9 | 11.5 | 14.8 | 16.5 |
| | 10% SCI | | | | | |
| | 0.5% Carbopol | | | | | |
| N | Comparative B: | 5 | 21.4 | 21.4 | 23 | 26.3 |
| | 10% SCI | | | | | |
| | 6% C16FA | | | | | |
| | 6% C18FA | | | | | |

### EXAMPLE 5

Two formulations as described in Table 4 were prepared as described below, where one (sample O) contains the inventive blend in Example 3 and the other (sample P) has only the fatty acid portion of the mixture (comparative case).

The inventive formula (O) was evaluated as having faster speed of rinsing, as indicated in Table 5, by a trained panel (21 panelists).

TABLE 4
Formulations

| | | | Inventive, | Comparative, |
|---|---|---|---|---|
| | | | Sample O | Sample P |
| TRADE NAME | | | % Active | % Active |
| Or chemical name | SUPPLIER | LOCATION | (Range) | (Range) |
| Amilite GCS-11 | Ajinomoto | Tokyo, Japan | 5-20 | 5-20 |
| Pationic 138 C | RITA Corp | Woodstock | 0-6 | 0-6 |
| | | Illinois | | |
| Disodium C16 | | | 3 | 0 |
| sulfosuccinate | | | | |
| Disodium C18 | | | 3 | 0 |
| sulfosuccinate | | | | |
| Prifrac 2960 | Uniqema | Netherlands | 1-3 | 1-3 |
| Kortaacid 1895 | Akzo Nobel | McCook, Illinois | 1-3 | 1-3 |
| Glycerine 99.7% USP | Dow Chemical | Bayonne, | 0-30 | 0-30 |
| | | New Jersey | | |
| DI WATER | | | to 100 | to 100 |

### Raw Materials

| TRADE NAME | SUPPLIER | LOCATION |
|---|---|---|
| Amilite GCS-11 | Ajinomoto | Tokyo, Japan |
| Jordapon CI-Prilled | PPG Industries | Gurnee, Illinois |
| Carbopol 981 | Noveon | Cleveland, Ohio |
| Prifrac 2960 | Uniqema | Netherlands |
| Kortaacid 1895 | Akzo Nobel | McCook, Illinois |
| Glycerine 99.7% USP | The Dow | Bayonne, |
| | Chemical | New Jersey |
| Pationic 138 C | RITA Corp | Woodstock, |
| | | Illinois |

TABLE 5
Rinsing speed

| | | | | d-prime** |
|---|---|---|---|---|
| | | | d-prime** | score on |
| | | d-prime score* | score on | “Speed of |
| | d-prime score* | on “bubble | “Speed of | rinsing off |
| | on “lather | adhesiveness | rinsing off | the slimy |
| Formulations | consistency” | to skin” | lather” | feel” |
| With | −1.7 | −1.0 | 0.1 | −0.3 |
| sulfosuccinates | | | | |
| (O) | | | | |
| Without | −3 | −2.3 | 1.4 | 0.7 |
| Sulfosuccinates | | | | |
| (P) | | | | |

*the higher the score, the better of lather attributes, i.e. more consistent lather and more adhesiveness to skin.
**the lower the score, the less time it takes to get a clean feel, and thus the faster rinsing speed

Methods:

Sample Preparation Methods

Samples A-G:

Sulfosuccinate and fatty acids were first mixed by means of stirring in desired amount of water at 75° C. (controlled by water bath) until no particulates are visible. An additional hour of mixing was followed to ensure complete homogeneity. The mixture, while being stirred, was then gradually cooled down in air to about 22 C.

Samples H-L & N:

Sulfosuccinate and fatty acids were first mixed by means of stirring in desired amount of water at 75° C. (controlled by water bath) until no particulates are visible. SCI powder of desired amount was then added carefully (to avoid foaming). After complete dissapearance of visible SCI flakes, an additional hour of mixing was followed to ensure complete homogeneity. The mixture, while being stirred, was then gradually cooled down in air to about 22 C.

Sample M:

Carbopol was first dissolved in desired amount of water at 75° C., then SCI powder was added carefully (to avoid foaming). After complete dissapearance of visible SCI flakes, an additional hour of mixing was followed to ensure complete homogeneity. The mixture, while being stirred, was then gradually cooled down in air to about 22 C.

O-P:

All ingredients except glycinate and lactylate were mixed at 75 C until no visible insolubles were present, then glycinate and lactylate were carefully added (to avoid foaming). An additional hour of mixing was provided after both glycinate and lactylate went into solution.

The mixture, while being stirred, was then gradually cooled down in air to about 22 C.

X-ray Diffraction

Small angle X-ray scattering was used to study the crystal/gel morphology. The instrument used has a standard Cu Kα radiation source with wavelength 1.54 Å, operating at 45 KV and 0.66 mA. Confocal Max Flux™ optics are used to focus the beam while ensuring high intensity. Scattered rays are recorded on a multi-wire, gas-filled 2-D detector (Reference: Practical X-ray Spectrometry, Jenkins, R. and De Vries, J. L., Springer-Verlag New York Inc., 1967), the scattering angles of which (the Q values) are calibrated using Silver Behenate powder (J. Appl. Cryst., Huang, TC., Toraya, H., Blanton, T N., Wu, Y., 26, 180-184, 1993). Test samples were equilibrated at about 22 C for 6 days prior to the measurement (no changes were detected for the current studied sulfosuccinate/fatty acid-only samples kept for a longer time). The samples were then loaded in a stainless steel cell having circular opening about 0.3 mm in thickness, 0.6 mm in diameter, and with mica as the window. The X-ray beam path was fully enclosed and vacuum generated to −1.0 bar. After gathering data on the 2-D detector, standard procedure was adopted to transform the 2-D spectrum into 1-D. Data interpretation followed well know methods (e.g. see Methods of X-ray and Neutron Scattering in Polymer Science, Ryong-Joon Roe, Oxford University Press, 2000).

Standard Viscosity Measurement

Scope:

This method covers the measurement of the viscosity of the ordered liquid crystalline cleansing composition.

Apparatus:

Rheometric Scientific SR-5 Stress Controlled Rheometer.

Procedure:

1. Turn on air and make sure pressure is between 90 and 110 psi.
2. Turn on temperature control and set to 25 C.
3. Mount the 25 mm cone (0.0994 radiant) to the upper fixture
4. Zero gap at a given normal force, e.g. 50 g
5. Set gap to 40 mm
6. Load test sample to the center of the bottom plate, use caution to avoid air entrainment
7. Set gap to 0.1 mm, and trim off the excess sample around the edge
8. Set gap to 0.0559 mm
9. Run steady shear sweep from 0.01 S⁻¹ to 10 S⁻¹, and record viscosity in Pa S.
   Water Loss Test

Samples were prepared as described above, and equilibrated at about 22 C for 4 days prior to the test (a longer time did not affect the results). Oven was preheated to 75 C. 20 g test sample, contained in a cylindrical glass vial (capacity: 30 g water) of 6.6 cm-high, 3 cm-diameter, and 2.5 cm-opening, was placed in the oven for desired period of time and then weighed (X). The percentage of water loss was calculated as:
(20−X)/(20*Y %)*100%, where Y is the water percentage in the initial sample.

Specific water loss is defined as the percentage of water loss after 60 minutes at 75 C.

Panel Evaluation On Rinse

The panel, consisting of 21 Japanese women between the ages 30 and 50, were trained and validated with products of different rinsing performances from slow slimy to fast squeaky. Other than water hardness, where 60 ppm Ca²⁺ water was used, there is no control over water temperature (estimated in the range 30-35 C) or washing habits. The panelists were asked to wash half of their face with a given standard (benchmark), and the other half of their face with the prototype. At the end of washing, i.e. after towel dry, panelists evaluated the relative magnitude of asked attributes. Recorded data was analyzed and presented as “d-prime” score. (D-prime (d′) is an estimate of a population value called Gamma which is a parameter in the Thurstonian model. The model is an application of Signal Detection Theory. The concept is that a stimulus evokes a response that is not a specific number but rather a point in a distribution. When 2 or more stimuli was presented, there are 2 or more distributions of response in a subject's mind. The differences between the mean of each response, drawn from the corresponding distribution, is the Gamma and its estimate is d′. The distance is measured in a unit of standard deviation. D-prime value is not method specific and it allows one to compare results from several studies. For more details, please refer to: Ennis, D. M., Thurstonian models for intensity ratings, IF Press, 2(3), 2-3). (1999). For this particular study, a minimum difference of 0.5 in d-prime is required to be perceivably different. For lather, a higher score means better quality lather. For rinse, a lower score means faster speed of rinse.

Lather Evaluation Method:

5.5 g of test formulation is weighed and transferred to the bottom of a graduated cylinder with the following dimensions: diameter 4.8 cm and length 42.0 cm. Then 30 g water was added at a temperature of 23 C to the cylinder very carefully to avoid any pre-dispersion of the formulation or foaming. The cylinder is rotated clock-wise and along its long-axis for desired number of revolutions at constant speed of about 38.6 rev./min, through an automated rotating device. Foam volume is read at the end of the rotation period.

Specific dispersion rate is defined as the volume of lather in mls per gram of test formulation generated after 40 rotations using the Lather evaluation method.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. An ordered liquid crystalline phase cleansing composition comprising:
a. at least about 3% by wt. of an anionic surfactant other than a C16 to C24 normal monoalkylsulfosuccinate;
b. a structuring base including at least one compound selected from C16 to C24 normal monoalkylsulfosuccinates and at least one compound selected from C16 to C24 normal alkyl fatty acids;
c. wherein the at least one monoalkylsulfocuccinate has an alkyl group with a carbon number n and the at least one normal fatty acid has an alkyl group with a carbon number m and the absolute value of n−m is 4 or less; and
d. wherein the mole ratio of said at least one monoalkylsulfosuccinate to said at least one fatty acid is in the range of about 3 to 0.2.

2. The cleansing composition of claim 1 wherein the said at least one normal monoalkylsulfosuccinate is present in a concentration of at least about 1% by wt.

3. The cleansing composition of claim 1 wherein the specific dispersion rate is at least about 40 mls when the viscosity of the composition is greater than about 100 Pa S at 25 C measured at a shear rate of 0.1 sec¹ according to the Standard viscosity method.

4. The cleansing composition of claim 1 wherein the pH is less than 8.0.

5. The cleansing composition of claim 1 wherein the solubilizing cation of the at least one monoalkylsulfosuccinate is selected from sodium, potassium, ammonium, alkanol ammonium or a blend thereof.

6. The cleansing composition of claim 1 wherein the composition has a specific water loss of less than about 10% by wt.

7. The cleansing composition of claim 1 wherein the composition has a lamellar, hexagonal, worm-like micellar or cubic surfactant phase or a combination thereof at 25 C.
